(19) United States
(12) Patent Application Publication
(10) Pub. No.: US 2017/0137784 A1
(43) Pub. Date: May 18, 2017

(54) NK Cell Culture Container And NK Cell Culture Method

(71) Applicant: CELLEX CORPORATION, Tokyo (JP)

(72) Inventor: Junichi Masuyama, Tochigi (JP)

(21) Appl. No.: 15/323,437

(22) PCT Filed: Mar. 23, 2015
(86) PCT No.: PCT/JP2015/058791

§ 371 Date: Jan. 3, 2017

(51) Int. Cl. C12N 5/0783; C12M 1/12

(52) U.S. Cl. CPC C12N 5/0646; C12M 25/16; C12N 2502/1171; C12N 2501/515; C12N 2501/599

(57) ABSTRACT

Provided are a culture vessel for NK cells and a method for culture of NK cells, which can provide high NK cell culture efficiency. The present invention has been completed by confirming that high NK cell culture efficiency can be achieved by a method of culturing a biological sample containing mononuclear cells in a cell culture vessel, a part or a whole of a surface of the cell culture vessel to be brought into contact with a biological sample being formed of a cycloolefin polymer, a part or a whole of the surface of the cell culture vessel to be brought into contact with a biological sample being coated with an anti-CD3 antibody and an anti-CD52 antibody.

### TECHNICAL FIELD

The present invention relates to a culture vessel for NK CELLS and a method for culture of NK cells.

### BACKGROUND ART

Cellular immunotherapies for cancer using autologous peripheral blood lymphocytes are mainly classified into αβT lymphocyte therapy, γδT lymphocyte therapy, NK cell therapy, NKT lymphocyte therapy, and the like depending on the kind of cells to be proliferated. In general, when lymphocytes are first strongly stimulated with antibodies, cytokines, or a certain kind of stimulating substance, the lymphocytes are activated to determine the direction of differentiation and to acquire a proliferation ability. The activated lymphocytes start to proliferate in the presence of a lymphocyte proliferation factor (interleukin 2 or the like) and continue dominant proliferation of a specific lymphocyte subset.

An anti-CD3 antibody is used to the first stimulation of αβT lymphocytes and then the αβT lymphocytes are proliferated sustainably in the presence of IL-2, and generally the proliferated αβT lymphocytes are used after two-week culture. In the earliest years, stimulation of lymphocytes with the anti-CD3 antibody was carried out using a plastic culture flask immobilized with the antibody, but had a risk of microbial contamination because of its complicated work.

In recent years, sealed polyethylene bags immobilized with the anti-CD3 antibody are commercially available and can induce stimulation and proliferation of αβT lymphocytes at a level equal to or higher than those induced using a flask. Further, the bags are provided as ready-to-use products, and hence are used widely as bags excellent in quality and safety.

There has been developed a method of dominantly inducing NK cells while suppressing unilateral proliferation of T lymphocytes by stimulating peripheral blood lymphocytes with two antibodies, an anti-CD3 antibody and an anti-CD52 antibody. This method can also induce NK cells using a polyethylene resin bag immobilized with the antibodies like the αβT lymphocytes.

### CITATION LIST

### Patent Literature

[PTL 1] JP 2005-058103 A

### SUMMARY OF INVENTION

### Technical Problem

The method of dominantly inducing NK cells by stimulating peripheral blood lymphocytes with two antibodies, an anti-CD3 antibody and an anti-CD52 antibody, can induce NK cells using a polyethylene resin bag immobilized with the antibodies like the αβT lymphocytes. However, when many cases are investigated, there is a considerable portion of them in which NK cells are induced at a low level. In some cases, the polyethylene resin bag has an excellent sealing property against microbial contamination, but often causes a low level of induction of NK cells, and hence it is necessary to develop a culture vessel that can improve the low level of induction of NK cells.

Meanwhile, stimulation of peripheral blood lymphocytes with the anti-CD3 antibody and the anti-CD52 antibody has heretofore been carried out using a plastic culture flask, but also in this method, there are some cases of a low ability to induce NK cells. This method has the above-mentioned problem of flask culture and requires a step of washing antibodies in a bag before addition of peripheral blood lymphocytes, and hence it is necessary to develop a method that can perform culture more easily and provides high NK cell induction efficiency.

### Solution to Problem

The inventors of the present invention have made extensive investigations in order to solve the above-mentioned problems. As a result, the inventors have confirmed that high NK cell culture efficiency (high NK cell induction efficiency) can be achieved by culturing a biological sample containing mononuclear cells in a cell culture vessel, a part or a whole of a surface of the cell culture vessel to be brought into contact with the biological sample being formed of a cycloolefin polymer, a part or a whole of the surface of the cell culture vessel to be brought into contact with the biological sample being coated with an anti-CD3 antibody and an anti-CD52 antibody. Thus, the present invention has been completed.

That is, the present invention includes the following.
1. A culture vessel for NK cells for use in culture of NK cells, a part or a whole of a surface of the culture vessel to be brought into contact with a biological sample being formed of a cycloolefin polymer, a part or a whole of the surface of the culture vessel to be brought into contact with a biological sample being coated with an anti-CD3 antibody and an anti-CD52 antibody.
2. A method for culture of NK cells, including: adding a biological sample containing mononuclear cells to a culture vessel, a part or a whole of a surface of the culture vessel to be brought into contact with a biological sample being formed of a cycloolefin polymer, a part or a whole of the surface of the culture vessel to be brought into contact with a biological sample being coated with an anti-CD3 antibody and an anti-CD52 antibody; and culturing the biological sample.
3. A method for culture of NK cells, including: adding a biological sample containing mononuclear cells to a culture vessel including beads having bound thereto an anti-CD3 antibody and an anti-CD52 antibody; and culturing the biological sample.
4. A method for culture of NK cells, including: adding a biological sample containing mononuclear cells to a culture vessel including beads having bound thereto an anti-CD3 antibody and an anti-CD52 antibody, a part or a whole of a surface of the culture vessel to be brought into contact with the biological sample being formed of a cycloolefin polymer; and culturing the biological sample.

### Advantageous Effects of Invention

The culture vessel for NK cells and the method for culture of NK cells using antibody-bound beads of the present invention can provide high NK cell culture efficiency.

### BRIEF DESCRIPTION OF DRAWINGS

FIG. 1 is a graph for showing results of comparison between culture of NK cells using a polyethylene culture bag and culture of NK cells using a cycloolefin polymer culture bag.

FIG. 2 is a graph for showing results of comparison between culture of NK cells using antibody-bound magnetic beads and culture of NK cells using an antibody-bound plastic culture plate.

### DESCRIPTION OF EMBODIMENTS

The present invention is directed to: a culture vessel for NK cells, a part or a whole of a surface of the culture vessel to be brought into contact with a biological sample being formed of a cycloolefin polymer, a part or a whole of the surface of the culture vessel to be brought into contact with a biological sample being coated with an anti-CD3 antibody and an anti-CD52 antibody; and a method for culture of NK cells using the culture vessel for NK cells. The culture vessel for NK cells and the method for culture of NK cells of the present invention are described below in detail.

(NK Cells)

Any NK cells acquired by a method known per se may be utilized as NK cells to be used in the present invention.

An NK cell donor and an NK cell recipient are preferably the same species. For example, when the donor is a human, the recipient is a human.

Further, the NK cell donor and the NK cell recipient are more preferably the same individual. For example, when the donor is a donor X, the recipient is the donor X.

(Culture of NK Cells)

The term “culture of NK cells” as used herein means differentiation, stimulation, mutation, induction, maintenance, proliferation, activation, and the like of NK cells, but is not particularly limited.

(Method for Acquisition of NK Cells)

The NK cells to be used in the present invention may be acquired by a method known per se (see: JP 5016732 B2). The NK cells are acquired by induction from mononuclear cells collected from peripheral blood, lymph nodes, thymus, bone marrow, tumors, pleural effusion, ascites, or umbilical cord blood, more preferably peripheral blood mononuclear cells.

For example, the mononuclear cells containing the NK cells may be collected from peripheral blood by a specific gravity centrifugation method.

(Method for Activation of NK Cells)

In a method for activation of NK cells of the present invention, mononuclear cells containing T lymphocytes and NK cells can be stimulated with a CD3 agonist (particularly an anti-CD3 antibody) and a CD52 agonist (particularly an anti-CD52 antibody) to activate the NK cells more than the T lymphocytes, and the NK cells can be proliferated safely and simply without being mixed with K562 and the like.

Particularly when the mononuclear cells containing T lymphocytes and NK cells are stimulated with the anti-CD3 antibody and the anti-CD52 antibody in the presence of IL-2, the NK cells can be more proliferated than the T lymphocytes as compared to stimulation with IL-2 alone. Further, the use of the method for activation of NK cells allows the NK cells to be proliferated 1,000 times or more (see: JP 2005-124568 A).

(Biological Sample)

The biological sample to be used in the present invention is not particularly limited as long as NK cells can be cultured by stimulation with an anti-CD3 antibody and an anti-CD52 antibody, but is exemplified by mononuclear cells collected from peripheral blood, lymph nodes, thymus, bone marrow, tumors, pleural effusion, ascites, or umbilical cord blood, more preferably peripheral blood mononuclear cells.

(Cycloolefin Polymer)

The cycloolefin polymer (which may be referred to as “cycloolefin” or “COP”) to be used in the present invention is formed of a composition containing COP as a major component (50 mass % or more, 60 mass % or more, 70 mass % or more, 80 mass % or more, or 90 mass % or more). The term “COP” means a polymer having a cyclic olefin structure.

In addition, the cycloolefin polymer is commercially available. For example, ZEONEX™ (ZEON Corporation) may be used.

(Anti-CD3 Antibody and Anti-CD52 Antibody)

The anti-CD3 antibody and anti-CD52 antibody to be used in the present invention are not particularly limited as long as NK cells can be cultured by bringing the anti-CD3 antibody and anti-CD52 antibody into contact with a biological sample. Further, the phrase “coating with the anti-CD3 antibody and the anti-CD52 antibody” means a state in which the anti-CD3 antibody and the anti-CD52 antibody are bound to a cycloolefin polymer or culture vessel surface to be brought into contact with a biological sample by any force (covalent bond, hydrogen bond, electrostatic interaction, hydrophobic interaction, or the like).

(Beads)

Beads to be used in the present invention are not particularly limited as long as the anti-CD3 antibody and the anti-CD52 antibody can be bound to the beads. The beads are particularly preferably magnetic beads because collection can be carried out easily.

(Culture Vessel for NK Cells)

In the culture vessel for NK cells of the present invention, at least a part or a whole of a surface to be brought into contact with a biological sample is formed of a cycloolefin polymer, and a part or a whole of the surface of the culture vessel to be brought into contact with a biological sample is coated with an anti-CD3 antibody and an anti-CD52 antibody.

The main body of a vessel including a biological sample, a culture medium, or the like may have any shape, but preferably has a bag shape in view of transportation, storage, or the like. The term “main body of a vessel” means a portion including a biological sample, a culture medium, or the like, but the main body per se of the vessel may be used as the culture vessel.

(Method for Culture of NK Cells)

The method for culture of NK cells of the present invention may be exemplified by the following methods.

(1) A method for culture, including: adding a biological sample containing mononuclear cells to a culture vessel, a part or a whole of a surface of the culture vessel to be brought into contact with a biological sample being formed of a cycloolefin polymer, a part or a whole of the surface of the culture vessel to be brought into contact with a biological sample being coated with an anti-CD3 antibody and an anti-CD52 antibody; and culturing the biological sample.

(2) A method for culture, including: adding a biological sample containing mononuclear cells to a culture vessel including beads having bound thereto an anti-CD3 antibody and an anti-CD52 antibody; and culturing the biological sample.

(3) A method for culture, including: adding a biological sample containing mononuclear cells to a culture vessel including beads having bound thereto an anti-CD3 antibody and an anti-CD52 antibody, a part or a whole of a surface of the culture vessel to be brought into contact with the biological sample being formed of a cycloolefin polymer; and culturing the biological sample.

The present invention is hereinafter specifically described by way of Examples. However, the present invention is not limited to these Examples.

### EXAMPLE 1

### Culture of NK Cells using Cycloolefin Polymer Bag

Culture of NK cells using a cycloolefin polymer bag was compared to culture of NK cells using a polyethylene resin bag. The details are as described below.

(Method)

An anti-CD3 antibody and an anti-CD52 antibody were separately immobilized on a polyethylene resin bag (TAZETTA (untreated bag), manufactured by Kohjin Bio Co., Ltd.) and a cycloolefin polymer bag (manufactured by Fukoku Co., Ltd.).

Specifically, 100 ng/ml of an anti-CD3 antibody (OKT3; manufactured by Janssen Pharmaceutical K.K.) and 20 μg/ml of an anti-CD52 antibody (MabCampath; manufactured by Sanofi K.K.) were added to PBS (manufactured by Kohjin Bio Co., Ltd.) to prepare antibody solutions.

Subsequently, 9 ml of the antibody solutions were separately added to both the bags each having an area adjusted to 90 cm².

After the addition, both the bags were left to stand still at from 4° C. to 8° C. for 24 hours and washed twice with 20 ml of PBS.

After the washing, 40 ml of a culture medium containing 4×10⁷ peripheral blood mononuclear cells (PBMCs) (Cellex NKGM-1; manufactured by Kohjin Bio Co., Ltd.) of a healthy subject, 4 ml of autologous plasma, and 500 U/ml of IL-2 were added to both the bags, followed by culture at 5% CO₂ and 37° C.

An adequate amount of the culture medium was further added to both the bags at an appropriate timing, and at the time point when many colonies were visually observed, the cells were transferred to a bag containing 1 L of Cellex NKGM-1 (manufactured by Kohjin Bio Co., Ltd.) and cultured for an additional 10 days while an adequate amount of the culture medium and IL-2 were further added thereto.

After the culture, 3×10⁵ proliferated lymphocytes were collected and stained with fluorescently labeled antibodies (anti-CD3 antibody and anti-CD56 antibody), and ratios of NK cells (CD3-CD56+) were detected using a flow cytometer and compared.

(Results)

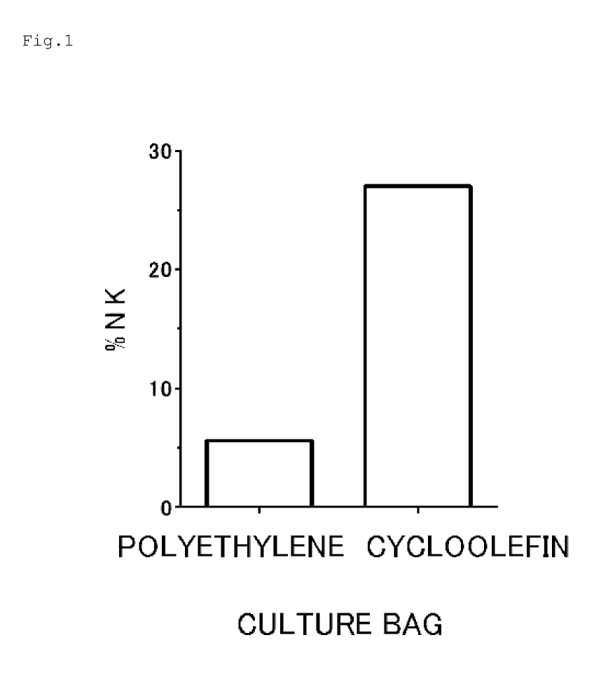

The results of detection of the ratios of the NK cells (CD3-CD56+) using the flow cytometer are shown in FIG. 1. As is apparent from the results shown in FIG. 1, the effect of the NK cell culture using the cycloolefin resin bag was found to be about five times as high as that of the NK cell culture using the polyethylene polymer bag.

### EXAMPLE 2

### Culture of NK Cells Using Antibody-Bound Magnetic Beads

Culture of NK cells using antibody-bound magnetic beads was compared to culture of NK cells using an antibody-bound plastic culture plate. The details are as described below.

(Method)

Dynabeads Tosylactivated M-450 (Dynal) were used as the magnetic beads. Specifically, 100 μl of the beads (4×10⁷ beads) were washed twice with PBS and suspended in 1 ml of PBS, and 100 ng/ml of an anti-CD3 antibody (OKT3; manufactured by Janssen Pharmaceutical K.K.) and 20 μg/ml of an anti-CD52 antibody (MabCampath; manufactured by Sanofi K.K.) were further added thereto, followed by stirring by rotation at room temperature for 24 hours. After the stirring, the anti-CD3 antibody- and anti-CD52 antibody-bound magnetic beads were washed twice with PBS and suspended in 200 μl of PBS supplemented with 0.1% bovine serum albumin to prepare an antibody liquid containing the anti-CD3 antibody- and anti-CD52 antibody-bound magnetic beads.

Subsequently, as a control, 0.4 ml of a liquid containing the above-mentioned concentrations of the antibodies and containing no magnetic beads was added to each well of a 24-well plastic culture plate (manufactured by Becton, Dickinson and Company, catalog No. 3047), and the plate was left to stand still at from 4° C. to 8° C. for 24 hours. Immediately before use of the plate, the plate was washed twice with PBS to prepare an antibody-bound plate.

Subsequently, 1 ml of a culture medium (Cellex NKGM-1; manufactured by Kohjin Bio Co., Ltd.) containing 1×10⁶ PBMCs derived from two healthy subjects having relatively low NK cell proliferation ability, 10% autologous plasma, and 500 U/ml of IL-2 was added to each well of a non-antibody-treated 24-well plate (the present invention) and an antibody-bound 24-well plate (control). 2.5 μl of antibody-bound magnetic beads (1×10⁶ beads) were added to PBMCs having been added to the non-antibody-treated plate. After the addition, the cells in the plates were cultured at 5% CO₂ and 37° C.

At the time point when many colonies appeared on day 3 or 4, one tenth of the liquid was transferred to a new plate, and 1 ml of the culture medium and 200 U/ml of IL-2 were added thereto.

Since then, for 10 days, the liquid was appropriately divided, and the culture medium and IL-2 were further added thereto.

On day 14 of culture, 3×10⁵ proliferated lymphocytes having been stimulated with the antibody-bound magnetic beads (the present invention) and the antibody-bound plate (control) were separately collected and stained with fluorescently labeled antibodies (anti-CD3 antibody and anti-CD56 antibody), and ratios of NK cells (CD3-CD56+) were detected using a flow cytometer and compared.

(Results)

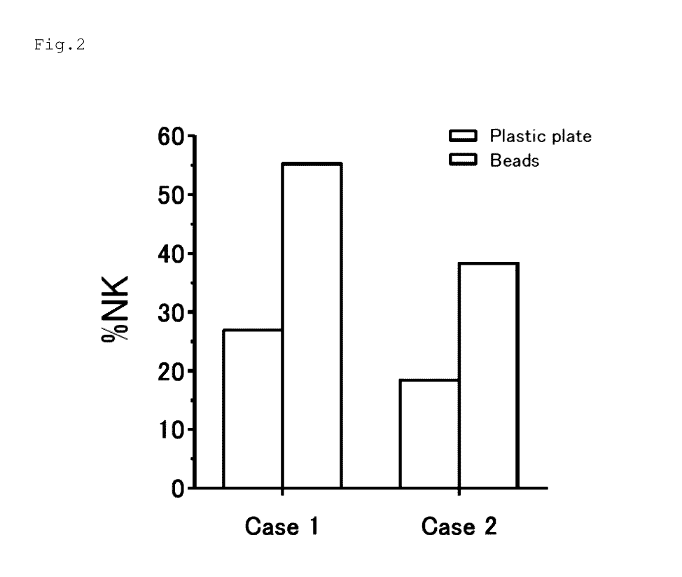

The results of detection of the ratios of the NK cells (CD3-CD56+) using the flow cytometer are shown in FIG. 2. As is apparent from the results shown in FIG. 2, the effect of the NK cell culture using the antibody-bound magnetic beads was found to be about twice as high as that of the NK cell culture stimulated with the antibody-bound plastic plate.

### INDUSTRIAL APPLICABILITY

The present invention can provide a highly efficient culture vessel for NK cells and a highly efficient method for culture of NK cells.

1. A culture vessel for NK cells for use in culture of NK cells,
a part or a whole of a surface of the culture vessel to be brought into contact with a biological sample being formed of a cycloolefin polymer,
a part or a whole of the surface of the culture vessel to be brought into contact with a biological sample being coated with an anti-CD3 antibody and an anti-CD52 antibody.

2-4. (canceled)

5. A culture vessel for NK cells for use in culture of NK cells,
a part or a whole of a surface of the culture vessel to be brought into contact with a biological sample being formed of a cycloolefin polymer,
wherein the culture vessel includes beads having bound thereto an anti-CD3 antibody and an anti-CD52 antibody.

6. A method for culture of NK cells, comprising:
adding a biological sample containing mononuclear cells to a culture vessel, a part or a whole of a surface of the culture vessel to be brought into contact with a biological sample being formed of a cycloolefin polymer, a part or a whole of the surface of the culture vessel to be brought into contact with a biological sample being coated with an anti-CD3 antibody and an anti-CD52 antibody; and culturing the biological sample,
or
adding a biological sample containing mononuclear cells to a culture vessel including beads having bound thereto an anti-CD3 antibody and an anti-CD52 antibody; and culturing the biological sample,
or
adding a biological sample containing mononuclear cells to a culture vessel including beads having bound thereto an anti-CD3 antibody and an anti-CD52 antibody, a part or a whole of a surface of the culture vessel to be brought into contact with the biological sample being formed of a cycloolefin polymer; and culturing the biological sample.
